(19) United States
(12) Patent Application Publication
(10) Pub. No.: US 2019/0062716 A1
(43) Pub. Date: Feb. 28, 2019

(54) METHOD FOR OBTAINING A PREPARATION OF BETA-AMYLASES FROM THE SOLUBLE FRACTIONS OF STARCH PLANTS

(71) Applicant: Roquette Freres, Lestrem (FR)

(72) Inventors: Vincent Courbois, Bethune (FR); Pierrick Duflot, La Couture (FR); Damien Passe, Douai (FR)

(21) Appl. No.: 16/174,536

(22) Filed: Oct. 30, 2018

Related U.S. Application Data
(63) Continuation of application No. 13/260,767, filed on Sep. 28, 2011.

(30) Foreign Application Priority Data
Mar. 30, 2009 (FR) 0951962

(51) Int. Cl. C12N 9/26

(52) U.S. Cl. CPC C12N 9/2425

(57) ABSTRACT

The present invention relates to a method for obtaining a preparation of β-amylases from the soluble fraction of starch plants, characterized in that the soluble fraction of starch plants is selected from the group consisting of the soluble fractions of wheat, pea, broad bean, horse bean, rice, barley, rye, buckwheat, potato and sweet potato, and preferably of wheat and barley, a clarification of said soluble fractions is carried out in such a way as to remove therefrom the insoluble substances and the colloids and, optionally, an ultrafiltration of said clarified soluble fractions is carried out in such a way as to obtain an ultrafiltration retentate containing the concentrated β-amylase and an ultrafiltration permeate, said ultrafiltration retentate containing the concentrated β-amylase is diafiltered and the resulting β-amylase is recovered.

The present invention relates to a method for obtaining a preparation of β-amylases from residues from the starch industry.

More particularly, the present invention relates to a method for obtaining a preparation of β-amylases from the liquid residues (also called “soluble fractions”, although they also contain residual insoluble substances, particles and colloids which are diverse and varied) produced during the wet extraction of the components (starches, proteins and fibers) of starch plants.

For the purpose of the invention, the term “starch plants” is intended to mean plants capable of being processed in the starch industry so as to extract the starch therefrom, such as, in particular, corn, potato, sweet potato, wheat, rice, pea, broad bean, horse bean, cassava, sorghum, konjac, rye, buckwheat and barley. In one particular embodiment, the “starch plants” comprise corn, potato, wheat, rice, pea, broad bean, horse bean, cassava, sorghum, konjac and barley.

The present invention also relates to said method comprising a first step of clarification of the soluble fractions and then, optionally, a second step of ultrafiltration so as to obtain a preparation that is purified (in the sense that it is freed of its salts) and/or concentrated with respect to β-amylases. In one preferred embodiment, the present invention also relates to said method comprising a first step of clarification of the soluble fractions, a second step of ultrafiltration, and then a third step of diafiltration so as to obtain a preparation that is purified (in the sense that it is depleted of salts) and concentrated with respect to β-amylases.

Finally, the present invention makes it possible to provide a preparation of β-amylases which is of a quality such that it is possible to use it in the same way as a purified β-amylase, in particular for the production of maltose-rich syrup.

β-Amylases are exohydrolases which release maltose units from the nonreducing ends of (α 1→4)-linked glucose polymers or oligomers, the reaction stopping at the first point of α 1→6 branching encountered.

Majority components of the “diastatic power” (corresponding to the combined activities of α-amylases, β-amylases, α-glucosidases and debranching enzymes) during malting (artificial germination of cereal seeds), the β-amylase activities isolated from this enzyme cocktail are essential for the production of maltose or of other fermentable sugars generated from starch.

The maltose and the other fermentable sugars thus obtained are, for example, intended for ethanolic fermentation by yeasts.

The saccharifying activity of β-amylases alone is therefore exploited in a large number of applications:
in bread making,
in the malt industry,
as a food additive, or even as a “digestive” agent,
for the production of maltose and of maltose-enriched syrups (precursor of maltitol and maltitol syrups),
for the production of sweeteners,
in pharmacy, for the production of vaccines.

The β-amylases of cereals, of legumes and of tubers are all similar with regard to their enzymatic activity.

However, the fact that some starch plants are selected with preference to others as sources of β-amylases for the malt industry, or for other biotechnological processes, depends on the richness of their seeds with respect to β-amylases, and depends on the ease with which they can be isolated from these media rich in varied enzymatic activities.

Thus, two sources of extraction of β-amylases are clearly identified.

The first source of extraction of “conventional” β-amylases is the endosperm of wheat, barley or rye, a source known to produce predominantly β-amylases in large amounts.

The second source of extraction corresponds to artificially germinated seeds. An artificial enrichment, in particular in β-amylases, is thus carried out through the triggering of germination; the β-amylase activities are then overexpressed with other amylase and glucosidase activities.

The technical field to which the present invention relates is that of the first source of extraction, making it possible to produce β-amylase as such more efficiently, in greater amounts. The technical field of the present invention also relates to the residual soluble fractions resulting from the processing of plants in the starch industry, such as wheat, barley, rye, corn, sorghum, buckwheat, rice, pea, broad bean, horse bean, potato, cassava, sweet potato or Konjac. The second source of β-amylases is more particularly intended for the preparation of enzyme cocktails.

It is already accepted by those skilled in the art that ungerminated barley, rye or wheat seeds are all biological materials of choice for the large-scale commercial preparation of β-amylases.

It is, moreover, known to those skilled in the art that half of the β-amylases that can be extracted from ungerminated barley, wheat or rye seeds can be readily obtained in the form of free enzymes by extraction with water and saline solutions.

The other half is partly in “bound” form which requires the addition of reducing agents or proteolytic enzymes for the extraction thereof.

Another β-amylase fraction that is not directly extractable, termed “latent” fraction, has also been described: detergents are necessary in order to extract it from cereal seeds.

Moreover, the β-amylase extraction methods described in the prior art are adapted according to the intended application.

More particularly, those intended for starch saccharification and for experimental tests are extracted intensively from botanical sources, such as soybean, wheat, barley, oats, rye and sweet potato, or are produced by microorganisms, such as Bacillus polymyxa and the like.

In the case of the preparation of maltose from starch, the first constraint is to limit as much as possible the concomitant production of other oligosaccharides, such as glucose and maltotriose. The second constraint consists in using a preparation of β-amylases which is substantially free of mycotoxins, of salts and of low-molecular-weight proteins and peptides (less than 50 kDa and preferably less than 30 kDa), and which is nonallergenic (in particular through the absence of bisulfite in the method of preparation), in order to allow rapid and easy purification (filtration, discoloration, demineralization) of the maltose-rich syrup and to obtain a more stable syrup.

In the malt industry, for example, it is known that the presence of glucose disturbs the fermentation of yeasts by inhibiting their growth.

For the commercial production of maltitol from maltose syrups, it is recommended to limit the production of sorbitol and maltotriitol.

Those skilled in the art will therefore prefer methods which make it possible to obtain β-amylase preparations that are as pure as possible. These methods will, of course, have the drawback of being expensive.

U.S. Pat. No. 3,492,203 thus describes the extraction of pure β-amylases from wheat bran for the preparation of maltose-rich syrup.

Starting the process from malt, which also contains α-amylase, which is difficult to separate from the β-amylase, is thus avoided.

The wheat bran then has the advantage of being relatively inexpensive, and recyclable as culture medium, but is especially selected for its predominant content of β-amylases.

The method consists in this case in extracting the pure β-amylase with water at a temperature between 20 and 40° C.

However, this method makes it possible to efficiently recover only half the available β-amylase content from the bran.

Moreover, the major drawback of this method is that it provides a β-amylase that is bacteriologically unstable: the β-amylase thus produced must be used extemporaneously for saccharifying the starch. No pasteurization is carried out.

In patent U.S. Pat. No. 3,801,462, the β-amylase is extracted from sweet potato, more particularly from the washing water after the step of rasping the sweet potatoes.

The actual method of extraction consists of a treatment at a pH of 4-4.5 and recovery of the β-amylase in the form of a precipitate. The extraction yield is higher when the temperature is adjusted to 55° C.

However, α-amylase and maltase activities still contaminate this β-amylase-enriched fraction, which of course complicates the obtaining of a pure preparation.

U.S. Pat. No. 4,675,296 proposes a method for preparing a commercial β-amylase by means of a method of extraction with water (at a temperature of between 5 and 50° C., for 5 to 70 h), but which requires starting from intact or partially dehusked barley grain.

The method described is tricky to implement since it is based on the use of the surface of the grain as a semipermeable membrane that would retain all the constituents of said grain, except the β-amylase.

It is, however, useful to add a reducing agent in order to facilitate the release of the β-amylase, said reducing agent being based on sulfur dioxide or sulfuric acid. Moreover, it is recommended to stabilize the enzyme with salts, polyols, sugars or acids.

Other methods in the literature recommend using enzymes in order to facilitate β-amylase extraction.

In international patent application WO 02/062980, β-amylases are thus extracted, in an aqueous medium, from cereals in the presence of a complex cocktail of enzymes having cellulase, hemicellulase and β-glucanase activities. The extract obtained must be finely purified in order to separate the β-amylase thus recovered from the reaction mixture.

The cereals selected are barley and wheat.

The extraction is carried out under reducing conditions (sulfite-containing water) in well-defined proportions, in clearly set temperature and reaction time ranges, and at pH 6.5.

In U.S. Pat. No. 3,769,168, the pathway of the purification of extracts enriched in β-amylases is preferred.

The crude extracts prepared from wheat bran, from soybean or from sweet potato are treated with an adsorbent of bentonite or kaolinite type.

The β-amylases adsorbed are then eluted using a solution which has an ionic strength over 0.5μ and a pH exceeding 5.

It follows from the foregoing that there remains an unsatisfied need to provide a simple and relatively inexpensive method for obtaining preparations of β-amylases of sufficient quality to allow in particular the efficient production of maltose-rich syrups.

The objective of the invention is therefore to meet this need, and the Applicant company has, to its credit, found, after numerous studies, that this objective can be achieved, against all expectations, as long as the residues from the starch industry, in particular the liquid residues from the starch industry (also called “soluble fractions”, although they still contain residual insoluble substances, particles and colloids which are diverse and varied) are used.

More particularly, the method for obtaining the preparation of β-amylases in accordance with the invention consists in selecting the media which are sources of β-amylases from the group consisting of the soluble fractions of wheat, pea, broad bean, horse bean, rice, barley, rye, buckwheat, sweet potato and potato, preferably the soluble fractions of wheat, pea, broad bean, horse bean, rice, barley and potato, more preferably the soluble fractions of wheat and barley, in clarifying said media and then, optionally, in carrying out an ultrafiltration in order to recover, or even concentrate, the β-amylases that they contain.

Preferably, the method for obtaining the preparation of β-amylases in accordance with the invention consists in selecting the media which are sources of β-amylases from the group consisting of soluble fractions of wheat, pea, broad bean, horse bean, rice, barley, rye, buckwheat, potato and sweet potato, preferably the soluble fractions of wheat and barley, in clarifying said media and then in carrying out an ultrafiltration and a diafiltration in order to recover and to concentrate the β-amylases that they contain. During the method for obtaining the preparation of β-amylases in accordance with the invention, no pH correction is carried out, the pH is naturally about 4.5.

It is to the Applicant company's credit to have found that the residues from the starch industry that are poorly exploitable in sectors other than that of fermentation (as nitrogenous source), or that of animal nutrition (mixed with products resulting from wheat) could constitute a medium of choice for readily extracting enzymes of interest, and more particularly β-amylases, at a level of quality sufficient for the targeted applications, such as the preparation of maltose-rich syrup.

The method in accordance with the invention is characterized in that:

a) the soluble fraction of starch plants is selected from the group consisting of the soluble fractions of wheat, pea, broad bean, horse bean, rice, barley, rye, buckwheat, sweet potato and potato, preferably wheat, potato, pea, broad bean, horse bean, rice and barley, and more preferably wheat and barley,
b) a clarification of said soluble fractions is carried out in such a way as to remove therefrom the insoluble substances and the colloids,
c) optionally, an ultrafiltration of said clarified soluble fractions is carried out in such a way as to obtain an ultrafiltration retentate containing the concentrated β-amylase and an ultrafiltration permeate,
d) the resulting concentrated α-amylase is recovered.

Preferably, the method in accordance with the invention is characterized in that:

a) the soluble fraction of starch plants is selected from the group consisting of the soluble fractions of wheat, potato, pea, broad bean, horse bean, rice, barley, rye, buckwheat and sweet potato, and preferably wheat and barley,
b) a clarification of said soluble fractions is carried out in such a way as to remove therefrom in particular the insoluble substances, the colloids and the microbiological material,
c) an ultrafiltration of said clarified soluble fractions is carried out in such a way as to obtain an ultrafiltration retentate containing the concentrated β-amylase and an ultrafiltration permeate (also called filtrate),
d) a diafiltration of said ultrafiltration retentate containing the concentrated β-amylase is carried out,
e) the resulting concentrated β-amylase is recovered.

The ultrafiltration permeate may also be mixed with the insoluble substances and colloids of step b).

The method in accordance with the invention therefore involves only the combination of physical extraction techniques, such as filtration or centrifugation techniques, and does not involve any enzymatic extraction step. There is therefore no need for the presence of an enzyme cocktail containing cellulase, hemicellulase and/or β-glucanase. There is also no need to carry out a pH correction.

The first step of the method in accordance with the invention consists in selecting the soluble fraction of starch plants from the group consisting of the soluble fractions of wheat, potato, pea, broad bean, horse bean, rice, barley, rye, buckwheat and sweet potato, preferably wheat, potato, pea, broad bean, horse bean, rice and barley, and more preferably wheat and barley.

In the present invention, the term “soluble fraction” is intended to mean the residual waters produced during the wet extraction of the noble components of starch plants. The term “noble components” is herein intended to mean the components exploited in the conventional starch industry, in particular, and according to the starch plant, the starch, whichever starch it is, the proteins, the fibers, the gums. Said soluble fraction according to the invention therefore comprises none, or substantially none, of these noble components. It is for this reason that these soluble fractions are also referred to as residual waters or alternatively liquid residues.

The soluble fractions of wheat or wheat solubles, for example, originate from the wheat “B”-starch separation stream resulting from the separation of starch in the wet wheat starch industry process. The B-starch or second starch is the starch consisting essentially of a predominant proportion of small starch granules or of damaged granules. According to the present invention, the wheat solubles consisting of the starch refining waters generally have about 5% of dry matter, in particular less than 8% of dry matter.

The soluble fractions of potato, sweet potato or cassava, or potato, sweet potato or cassava solubles, are obtained by recovery of the soluble fraction resulting from the tuber crushing at the beginning of starch extraction.

The soluble fractions of pea or pea solubles result from the pea steep water and are recovered before crushing and separation of the various pea constituents.

The soluble fractions of broad bean or broad bean solubles result from the broad bean steep water and are recovered before crushing and separation of the various broad bean constituents.

The soluble fractions of horse bean or horse bean solubles result from the horse bean steep water and are recovered before crushing and separation of the various horse bean constituents.

The soluble fractions of rice or rice solubles result from the rice steep water and are recovered before crushing and separation of the various rice constituents.

The soluble fractions of barley, rye or buckwheat, or barley, rye or buckwheat solubles, result from the “B”-starch separation stream resulting from the separation of starch in the barley starch industry process. The B-starch or second starch is the starch consisting essentially of a predominant proportion of small starch granules or of damaged granules.

These soluble fractions also contain high-molecular-weight proteins, some of which have enzymatic activities of interest, but which are, however, mixed with insoluble debris of all types (in particular fibers, traces of starch, etc.), colloidal substances (in particular phospholipids, glycoproteins, cells of the external layer of the endosperm, also called the aleurone layer, etc.) and microbiological material (microorganisms, bacteria, etc.).

The Applicant company was indeed up against a prejudice of the prior art which described, up until now, the use by those skilled in the art of these soluble fractions only in two fields of application with a low added value:
a source of nitrogen in fermentation, after a protease treatment has made the residual high-molecular-weight protein component assimilable by microorganisms,
a nutritive source for livestock animals, and further added to with fibrous matter (e.g. wheat bran in the case of wheat solubles).

The second step of the method in accordance with the invention therefore consists in carrying out a step of clarification of said soluble fractions in such a way as to remove therefrom the insoluble substances and the colloids.

This clarification step is verified by measuring the turbidity of the solution after each of the treatments carried out.

Turbidity denotes the content of a liquid in terms of matter which makes it cloudy. It is caused by colloidal particles which absorb, scatter and/or reflect light.

This turbidity measurement is carried out using a laboratory nephelometer (or tubidimeter).

This method is standardized (NF EN ISO 7027); two units of measurement of turbidity using formazine as standard:
FNU (Formazine Nephelometric Unit), or NFU used in decree No. 2001-1220 of Dec. 20, 2001. This unit measures the turbidity at an angle of 90° at a wavelength of 860 nm;
FAU (Formazine Attenuation Unit) measures transmitted light (180°).

The turbidity unit recommended by the Environmental Protection Agency (EPA—USA) is the NTU (Nephelometric Turbidity Unit). The measurement is carried out on light scattered at 90°, but at a wavelength different than 860 nm.

With regard to the measurement of the enzymatic activity, it consists in determining the diastase activity, a measurement conventionally used to determine the amylase activity of barley malt or of other enzymatic preparations (cf. article DIASTASE ACTIVITY—DIASTATIC POWER—of the section General Tests and Assay/Appendix V/pp 1117-1118 of the Food Chemical Codex 6).

The diastase activity is expressed in degrees of diastatic power (°DP), defined as the amount of enzyme contained in 0.5 ml of a 5% solution of a sample of enzyme preparation sufficient to reduce 5 ml of Fehling liquor, when said sample is placed in 100 ml of the substrate for 1 h at 20° C.

Since the diastatic power makes it possible to measure all the enzymatic activities of amylase type, the applicant company made sure by means of other, more specific enzymatic methods (for example using the Megazyme assay kit specific for α-amylase, sold by Ceralpha Method) that the preparation of β-amylases in accordance with the invention did not contain other contaminating activities.

Likewise, as will be exemplified hereinafter, the saccharification tests in the laboratory with the enzymatic preparation in accordance with the invention showed a high maltose content without any significant presence of glucose.

The first step of clarification of the method in accordance with the invention can be carried out in three different ways:

1) In a first embodiment of the method in accordance with the invention, the clarification of the soluble fractions is carried out by filtration, by means of a technique selected from the group consisting of frontal filtration, filtration under pressure and vacuum filtration.

The starting soluble fractions have a high turbidity, which cannot be measured given their high load of colloidal particles and residual insolubles.

In the case of the use of a filter under pressure, these soluble fractions are pushed through this filter preloaded with microcrystalline cellulose. The product exiting the filter then has a turbidity of about 20 to 60 NTU according to the type of cellulose used.

In the case of vacuum filtration, the Applicant company recommends carrying out the filtration using belt filters or rotary filters.

A prelayer filter under vacuum, preloaded with a layer of cellulose, is, for example, used.

2) In a second embodiment of the method in accordance with the invention, the clarification of the soluble fractions is carried out by centrifugation.

The supernatant from centrifugation of the solubles then also has a turbidity of 20 to 60 NTU according to the flow rate and the concentration of the centrifugation pellet (the higher the acceleration of the centrifuge, the lower the turbidity of the product exiting in the supernatant).

More particularly, the Applicant company recommends carrying out this centrifugation step using:
a disk centrifuge of NA7 self-cleaning or nozzle separator type, or
a disk centrifuge of BTUX high-performance vortex nozzle separator type (these two types of centrifuge, NA7 or BTUX, being sold, respectively, by the companies Westfalia and AlfaLaval).

After centrifugation, but optionally, it may useful to carry out an additional “safety” filtration with a view to reducing as much as possible the load of the soluble fractions. The filter selected is a Padovan® horizontal plate pressure filter.

The filter is loaded with a prelayer of cellulose of fine particle size.

The soluble fractions thus filtered are added to by means of a more or less large top-up with cellulose.

The turbidity of the filtrate thus falls to a value of between 5 and 10 NTU.

3) In a third embodiment of the method in accordance with the invention, the clarification of the soluble fractions is carried out by tangential membrane filtration.

More particularly, the Applicant company recommends carrying out the membrane filtration by tangential microfiltration with membranes, for example ceramic membranes, having a porosity of from 0.1 to 1 μm.

The permeate in this case advantageously has a turbidity of about 0 NTU.

However, this technique can result in a loss of β-amylases which adsorb to the membrane. However, this loss is negligible with regard to the amount initially present in the soluble fractions.

This clarification step of the method in accordance with the invention can consist of any one of these three embodiments (filtration, centrifugation or tangential membrane filtration) or of any combination of two or three of these embodiments.

Optionally, this clarification step may be preceded by a step of flocculation of the colloidal particles, by any technique known, moreover, to those skilled in the art, as will be exemplified hereinafter.

In order to have a preparation freed of the contaminating residual salts and to concentrate said preparation with respect to β-amylases, an ultrafiltration of said clarified soluble fractions is carried out in such a way as to obtain an ultrafiltration retentate containing the β-amylase and an ultrafiltration permeate. The ultrafiltration retentate is then dialyzed at constant volume in such a way as to reduce the concentration of impurities in said retentate.

More particularly, the Applicant company recommends carrying out the ultrafiltration using membrane having a cut-off threshold of from 10 000 Da to 50 000 Da, preferably a cut-off threshold of 30 000 Da (also denoted 30 kDa).

The soluble fractions are ultrafiltered on a module equipped with polysulfonated membranes having a cut-off threshold of 30 000 Da in cassettes on a laboratory scale and polysulfonated spiral membranes having a cut-off threshold of 30 000 Da on a pilot scale.

The enzyme becomes concentrated in the retentate as the volume concentration factor (VCF) increases.

For ten liters of a starting solution quantitatively determined as being at 22°DP/ml, two liters of retentate at 110°DP/ml and eight liters of permeate having no diastatic power are obtained for a VCF of 5.

The ultrafiltration retentate is diafiltered at constant volume, according to the method in accordance with the invention. This is because the obtaining of macromolecules with a high degree of purity requires the implementation of such a diafiltration step in order to strip (by dilution and permeation), from the ultrafiltration retentate, solutes not retained by the membrane.

A final step of the method in accordance with the invention may consist in mixing the ultrafiltration permeate with the insolubles and colloids of step b). This mixture is reunited with the untreated soluble fractions from the starch industry.

As will be exemplified, and surprisingly and unexpectedly, the preparation thus prepared has a quality such that it is possible to use it as it is, without additional treatment, as a source of enzymes intended for the preparation of maltose-rich syrups.

### EXAMPLE 1: OBTAINING A FIRST PREPARATION OF β-AMYLASES from Wheat Solubles—Laboratory Scale

In the manufacture of starch from wheat, 60 liters of soluble fractions are collected at the inlet of the solubles evaporator, a step conventionally carried out for cattle feed after concentration, sold by the Applicant company under the name Corami®.

These soluble fractions have a pH of 4 and a β-amylase activity of about 25° DP/ml.

These 60 liters are centrifuged in an SA1 desludging disk centrifuge sold by the company Westfalia, at a flow rate of 40 liters/hour.

45 liters of supernatant from the centrifugation are recovered. A turbidity of 60 NTU is measured.

They are then filtered on a Choquenet laboratory chamber sheet filter of 56 cm², a cloth filter preloaded with Clar-O-Cel 13/6 (CECA) and Vitacel L10 (J. Rettenmaier und Söhne) celluloses.

Continuous topping-up is also carried out with these celluloses at a rate of 1 g/l.

40 liters of filtrate having a turbidity of 8 NTU and a β-amylase activity of 22° DP/ml are recovered.

The filtrate is then ultrafiltered on a Millipore laboratory module with 0.18 m² of membranes.

39.5 liters of ultrafiltered filtrate and a retentate concentrated by a factor of 75, having a β-amylase activity of between 1500 and 1600° DP/ml, are recovered.

The ultrafiltration retentate is dialyzed at constant volume with 2.5 volumes of water continuously in such a way as to reduce the concentration of impurities of the solubles by a factor of 10.

The resulting preparation of β-amylases is then stored at +4° C.

### EXAMPLE 2: OBTAINING A FIRST PREPARATION OF β-AMYLASES FROM WHEAT SOLUBLES—LABORATORY SCALE WITH FLOCCULANT

In the manufacture of starch from wheat, 120 liters of soluble fractions are collected at the inlet of the solubles evaporator at a pH of 4.3.

They are then treated with 20 ppm of Fe³⁺ and 25 ppm of polymeric Flopam AN 923 PWG anionic flocculant (SNF) in such a way as to flocculate the insoluble and colloidal particles.

The soluble fractions thus treated are then centrifuged under the conditions of example 1 at a flow rate of 40 liters/hour.

90 liters of soluble fractions thus centrifuged are obtained. They have a turbidity of about 17 NTU and a β-amylase activity of 21° DP/ml.

In the same way as in example 1, this fraction is subjected to fine filtration on a chamber sheet filter.

80 liters of a filtered solution having a turbidity of 5 NTU are obtained.

Ultrafiltration is then carried out on a Millipore laboratory module with 0.18 m² of membranes having a cut-off threshold of 30 kDa.

79 liters of ultrafiltered filtrate and a retentate concentrated by a factor of 80 and having a β-amylase activity of between 1500 and 1600° DP/ml are recovered.

The ultrafiltration retentate is dialyzed at constant volume with 2.5 volumes of water continuously in such a way as to reduce the concentration of impurities of the solubles by a factor of 10.

The resulting preparation of β-amylases is then stored at +4° C.

### EXAMPLE 3: OBTAINING A FIRST PREPARATION OF β-AMYLASES FROM WHEAT SOLUBLES—PILOT SCALE

The pilot test consists of a test in which 10 m³ of soluble fractions of wheat are collected and prepared as in example 2.

The insoluble and colloidal particles are removed by centrifugation on an NA7 centrifuge in self-cleaning operating mode.

7 m³ of supernatant having a turbidity of 22 NTU are recovered.

This supernatant is subsequently filtered by frontal filtration on a 0.3 m² AMAFILTER filter loaded with the previously defined celluloses. It is topped up with 1 g/l of cellulose.

Once filtered, the solution has a turbidity of 5 NTU.

It is ultrafiltered on equipment consisting of polysulfonated spiral membranes having a cut-off threshold of 30 kDa.

75 liters of retentate concentrated by a factor of approximately 95 and having a β-amylase activity of approximately 2000° DP/ml are then obtained.

The ultrafiltration retentate is dialyzed at constant volume with 2.5 volumes of water continuously in such a way as to reduce the concentration of impurities of the solubles by a factor of 10.

In order to stabilize the enzyme, 50 kg of sorbitol powder sold by the Applicant company under the trademark Neosorb®, containing 99.5% of dry matter, are added.

The final preparation then has a β-amylase activity of 1550° DP/ml.

### EXAMPLE 4: OBTAINING THE PREPARATION OF β-AMYLASES FROM BARLEY SOLUBLES

100 kg of barley flour and 72 liters of water are added to a kneading machine so as to obtain a hard dough.

After standing at ambient temperature for 30 minutes, the dough is washed in countercurrent manner, in a rotary sieve made by the applicant company, with water so as to separate the starch from the gluten.

200 liters of a suspension of starch are collected, and are sieved on a first vibrating sieve of 100 μm, then a second of 63 μm, in order to remove the fibers, the gums and the residual gluten.

The filtrate is then centrifuged at high flow rate (200 l/h) on a decanter centrifuge sold under the name SEDICANTER by the trademark FLOTTWEG in such a way as to isolate the A-starches (large granule) in the concentrate and the B-starches (small granule) in the supernatant.

The B-starch suspension overflow is again centrifuged on the desludging SA1 diskseparator (WESTFALIA) at reduced flow rate (40 l/h) in such a way as to isolate the B-starch in the concentrate, and soluble fraction, in the supernatant. The pH is 4.5.

All these prior steps make it possible to obtain a soluble fraction in accordance with the present invention, i.e. freed of the noble components, in particular freed of the starch, whichever starch it is, the proteins, the fibers and the gums. 60 liters of said soluble fraction, which has a turbidity of 120 NTU, are taken.

The soluble fraction is treated according to the flocculation method of example No. 2.

Centrifugation is carried out on the previous desludging centrifuge at 40 l/h and 40 liters of soluble fraction having a β-amylase activity of 28°DP/ml and a turbidity of 21 NTU are recovered.

This soluble fraction is filtered on a chamber sheet filter preloaded with Clar-O-Cel 13/6 (CECA) and Vitacel L10 (J. Rettenmaier und Söhne) celluloses.

The soluble fraction was topped up with 1 g/l of celluloses.

40 liters of filtrate having a turbidity of 6 NTU and a β-amylase activity of 27° DP/ml are thus obtained.

Ultrafiltration is carried out on a Millipore laboratory module with 0.18 m² of 30 KDa membranes, described in example 2.

Concentration by a factor of approximately 65 is carried out.

0.6 liters of a solution of concentrated enzymes having a β-amylase activity of 1700°DP/ml is finally obtained.

The ultrafiltration retentate is dialyzed at constant volume of 2.5 volumes of water continuously in such a way as to reduce the concentration of impurities of the solubles by a factor of 10.

It is stabilized with 400 g of sorbitol powder sold by the Applicant company under the trademark Neosorb® containing 99.5% of dry matter.

The enzymatic preparation then has a β-amylase activity of 1400° DP/ml.

### EXAMPLE 5: OBTAINING MALTOSE-RICH SYRUPS, WITH A COMMERCIAL PURIFIED β-AMYLASE AS CONTROL

### a) Obtaining the Maltose Syrup Through the Action of β-Amylase Alone

A compared saccharification is carried out in the laboratory using a commercial purified β-amylase from the company GENENCOR (OPTIMALT BBA) and using the preparation of β-amylases in accordance with the invention, prepared according to example 1.

The β-amylase, at the concentration of 4% (commercial enzyme on dry starch), is added to a solution of liquefied starch containing 30% of dry matter, having a Dextrose Equivalent level of 6 (dissolution of maltodextrin of DE 6).

The doses are intentionally high in order to detect the possible presence of parasitic enzymatic activities.

The reaction is carried out at a temperature of 58° C. and at pH 5 for 24 hours.

The results are expressed as % of glucose monomers and oligomers generated by the β-amylase action, oligomers having a degree of polymerization equal to 2 and more, determined by High Pressure Liquid Chromatography of sodium type.

The DP2s correspond to maltose, the DP1s to glucose and the DP3s to maltotriose.

| | DP > 3 | DP3 | DP2 | DP1 |
|---|---|---|---|---|
| Starting solution | 96.5 | 1.5 | 1.2 | 0.3 |
| (t = 0) | | | | |
| GENENCOR β-amylase | 44 | 5.3 | 49.9 | 0.4 |
| (t = 24 h) | | | | |
| (β-Amylase example 1 | 42.6 | 5.7 | 51.4 | 0.4 |
| (t = 24 h) | | | | |

The results demonstrate that the preparation of β-amylases in accordance with the invention can effectively replace the commercial β-amylase.

b) Obtaining a Maltose-Rich Syrup Through the Action of a β-Amylase Preparation Combined with a Pullulanase and a Maltogenase in the Laboratory

The tests were carried out under the previous conditions, with the difference that 2 enzymes are added simultaneously with the addition of the β-amylase preparation.

They are the PULLUZYME® pullulanase sold by the company ABM, which specifically hydrolyzes the α 1→6 linkages of starch, and the maltogenic α-amylase sold by the company NOVOZYMES under the name MALTOGENASE, which specifically hydrolyzes the α 1→4 linkages.

These enzymes are also added in proportions of 4% (commercial enzyme on dry starch).

As a control, a β-amylase preparation originating from a malt extract (=cocktail of enzymes predominantly composed of β-amylases) is used as β-amylase source.

| | DP | | | |
|---|---|---|---|---|
| | GREATER | DP3 | DP2 | DP1 |
| Starting solution | 96.5 | 1.5 | 1.2 | 0.3 |
| (t = 0) | | | | |
| GENENCOR β-amylase | 7 | 2 | 85.3 | 5.5 |
| (t = 24 h) | | | | |
| β-Amylase according | 7.2 | 1.5 | 85.5 | 5.2 |
| to example 1 | | | | |
| (t = 24 h) | | | | |
| β-Amylase extracted | 6.3 | 3.3 | 83.3 | 7.0 |
| from malt T | | | | |
| (t = 24 h) | | | | |

While the carbohydrate profile obtained using the purified β-amylase and the carbohydrate profile obtained using the preparation of β-amylases in accordance with the invention are equivalent, thereby demonstrating the excellent behavior of said preparation according to the invention, the carbohydrate profile obtained using malt extract produces a much higher glucose content.

c) Obtaining a Maltose-Rich Syrup Through the Action of a β-Amylase Preparation Combined with a Pullulanase and a Maltogenase on the Industrial Scale

The preparation of example No. 3 is used to carry out the industrial test.

The saccharification is carried out on 200 m³ of a solution of liquefied starch containing 30% of dry matter, having a Dextrose Equivalent level of 6. The β-amylase preparation (1% on a dry basis), the pullulanase and the maltogenase are added simultaneously under the following conditions: pH 5, 55° C.

The following results are obtained after 66 hours (expressed as % DP generated−measurement carried out by sodium HPPLC):

| DP > 3 | DP3 | DP2 | DP1 |
|---|---|---|---|
| 6 | 1.1 | 88.3 | 4.3 |

This maltose-rich syrup can be purified, hydrogenated and crystallized so as to obtain a maltitol of quality identical to that obtained by the conventional routes.

### d) Obtaining Maltose Syrup Through the Action of the Ultrafiltered and Diafiltered β-Amylase Preparation

A compared saccharification is carried out in the laboratory using the preparation of β-amylases in accordance with the invention, prepared according to example 1 (β-amylase example 1), and using a preparation of β-amylases clarified according to example 1 but not subjected to the ultrafiltration and diafiltration steps (β-amylase without UF).

For the saccharification with the “β-amylase example 1” preparation, said “β-amylase example 1” preparation is added, at the concentration of 4% (commercial enzyme on dry starch), to a solution of liquefied starch containing 30% of dry matter, having a Dextrose Equivalent level of 6 (dissolution of maltodextrin of DE 6). The enzymatic preparation then has a β-amylase activity of 1.8°DP/ml. The reaction is carried out at a temperature of 58° C. and at pH 5 for 24 h.

For the saccharification with the “β-amylase without UF” preparation, l 1 of filtrate at 8 NTU of example 1 (filtrate neither ultrafiltered nor diafiltered) was taken. Said filtrate at 8 NTU is diluted to 1/15^{th} so as to obtain a concentration of β-amylase at 1.8°DP/ml. The pH is adjusted to 5. 700 ml of said diluted filtrate are taken and 300 g of maltodextrin of DE 6 are added. The reaction is carried out at a temperature of 58° C. for 24 h.

The following results are obtained (expressed as % DP generated−measurement carried out by sodium HPPLC):

| | DP > 3 | DP3 | DP2 | DP1 |
|---|---|---|---|---|
| Starting solution | 96.5 | 1.5 | 1.2 | 0.3 |
| (t = 0) | | | | |
| β-Amylase without UF | 41.5 | 3.8 | 48.5 | 2.2 |
| (t = 24 h) | | | | |
| β-Amylase example 1 | 42.6 | 5.7 | 51.4 | 0.4 |
| (t = 24 h) | | | | |

The results demonstrate that the nonultrafiltered and nondiafiltered preparation of β-amylases (β-amylase without UF) makes it possible to obtain a maltose-rich syrup containing 2.2% of glucose. Unlike the “β-amylase example 1” preparation (maltose-rich syrup obtained containing 0.4% of glucose), the “β-amylase without UF” preparation is therefore contaminated with enzymatic impurities.

In order to evaluate the degree of purity of the maltose-rich syrups obtained using the two preparations, “β-amylase example 1” and “β-amylase without UF”, in particular the amount of salts that they contain, the conductivity of said syrups was measured at 20° C. (CDM210 instrument sold by the company Mettler) after filtration of the syrups on a disc filter with a porosity of 1 μm from the company Millipore.

The results are the following:

| | Conductivity (μS) |
|---|---|
| β-Amylase without UF | 200 |
| β-Amylase example 1 | 20 |

The ultrafiltered and diafiltered preparation (“β-amylase example 1”) makes it possible to obtain a maltose-rich syrup which contains a much lower amount of salts than the maltose-rich syrup obtained by means of the “β-amylase without UF” preparation. Consequently, the purification (filtration, discoloration, demineralization) of the syrup obtained by means of the “β-amylase without UF” preparation will be more difficult than that of the syrup obtained by means of the “β-amylase example 1” preparation.

1-10. (canceled)

11. A method for obtaining a preparation of β-amylases from the soluble fraction of starch plants comprising:
a) clarification of a soluble fraction selected from the group consisting of soluble fractions of wheat, pea, broad bean, horse bean, rice, barley, rye, buckwheat, sweet potato and potato said clarification comprising the removal of insoluble substances and colloids;
b) an optional ultrafiltration of said clarified soluble fraction to obtain an ultrafiltration retentate containing concentrated β-amylase and an ultrafiltration permeate; and
c) recovery of the resulting concentrated β-amylase.

12. The method of claim 11, wherein step b) is carried out, and that, between steps b) and c), a diafiltration of said ultrafiltration retentate containing the concentrated β-amylase is carried out.

13. The method of claim 11, wherein the clarification of the soluble fraction is carried out by a filtration technique selected from the group consisting of frontal filtration, filtration under pressure and vacuum filtration.

14. The method of claim 13, wherein vacuum filtration is carried out using belt filters or rotary filters.

15. The method of claim 11, wherein said clarification is carried out by centrifugation.

16. The method of claim 15, wherein the centrifugation is carried out using a disk centrifuge of self-cleaning or nozzle separator type, or a disk centrifuge of high performance vortex nozzle separator type.

17. The method of claim 11, wherein said clarification is carried out by tangential membrane filtration.

18. The method of claim 17, wherein the tangential member filtration is a microfiltration having a cut-off threshold of between 0.1 and 1 rm.

19. The method of claim 11, wherein the ultrafiltration is carried out using a membrane having a cut-off threshold of from 10,000 Da to 50,000 Da.

20. A method of preparing maltose-rich syrups comprising contacting a solution of liquefied starch with a β-amylase composition purified according to the method of claim 11 under conditions that permit saccharification of said solution of liquefied starch.
